(19) United States
(12) Patent Application Publication
(10) Pub. No.: US 2008/0154299 A1
(43) Pub. Date: Jun. 26, 2008

(54) Forceps for performing endoscopic surgery

(76) Inventor: Steve Livneh, Amherstburg (CA)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 11/953,709

(22) Filed: Dec. 10, 2007

Related U.S. Application Data
(60) Provisional application No. 60/873,718, filed on Dec. 8, 2006.

(51) Int. Cl. A61B 17/28

(52) U.S. Cl. 606/205

(57) ABSTRACT

A surgical instrument for performing surgery includes a housing defining an axial bore. A lock is slidably disposed in the transverse bore to engage and disengage a tube adaptor. A latch extends from the lock to move the latch into and out of the axial bore. The lock and latch allow for interchangeability of tip assemblies inserted into the surgical instrument. A first and second handle are pivotally attached to the housing. A first ratchet member is rotatably attached to the first handle and includes serrated teeth. A second ratchet member extends from the second handle. The second ratchet member including a finger for engagement with the serrated tooth to prevent movement of said first handle relative to the second handle. The first ratchet member is rotatable about the rotational axis to disengage from the second ratchet member and allow the first handle to move relative to the second handle.

### CROSS-REFERENCE TO RELATED APPLICATIONS

The subject patent application claims priority to, and all the benefits of, U.S. Provisional Patent Application Ser. No. 60/873,718 which was filed on Dec. 8, 2006, the entire specification of which is expressly incorporated herein by reference.

### BACKGROUND OF THE INVENTION

1. Field of the Invention

The invention generally relates to forceps for performing endoscopic surgery. More specifically, the present invention relates to forceps which are configurable for performing different endoscopic procedures.

2. Description of the Related Art

Today's endoscopic and arthroscopic surgical instruments encompass a multitude of different designs. While all may be designed to serve the same function, each one may be shaped differently to provide the surgeon better access to perform the procedure. For example, a pair of forceps may include a tube that extends from a pair of handles. A blade is disposed at an end of the tube for performing the surgery. The tube, near the blade in one pair of forceps is bent upward to provide the surgeon with the required access in the patient to make a first cut. However, if the surgeon needs to perform a second cut, on the same patient, but in a different position, the surgeon must get a different pair of forceps where the tube is bent to a different orientation. Additionally, if the surgeon needs to move the handles to perform a scissor motion with the blade and then use the blade to grasp, the surgeon must get a different pair of handles.

Based on the above, it is easy to relate to today's realities of the operating room where a large inventory of specific instruments must be kept in an inventory at a high cost. Managing and maintaining this inventory is costly and complex. Lack of flexibility among the instruments are a direct added cost to each surgery, while maintaining the requisite variety of instruments necessitates added personnel and sterilization facilities and capabilities.

### SUMMARY OF THE INVENTION

In a first aspect, the present invention provides a body assembly of a surgical instrument for performing endoscopic surgery. The handle assembly includes a housing defining an axial bore. The housing defines a transverse bore which extends along a transverse axis and opens to the axial bore. A lock is slidably disposed in the transverse bore, along the transverse axis, for sliding into the axial bore to engage a tube adaptor and for sliding out of the axial bore to disengage the tube adaptor. A latch extends from the lock to an end portion. The latch defines a pivot axis for pivoting the latch about the pivot axis as the lock slides into or out of the axial bore.

In a second aspect, the present invention provides a surgical instrument for performing endoscopic surgery. The surgical instrument includes a housing defining an axial bore extending along a tube axis. The housing defines a transverse bore extending along a transverse axis and opening to the axial bore. A lock is slidably disposed in the transverse bore along the transverse axis for sliding into the axial bore to engage a tube adaptor and for sliding out of the axial bore to disengage the tube adaptor. A latch extends from the lock to an end portion with the latch defining a pivot axis for pivoting the latch about the pivot axis as the lock slides into or out of the axial bore. A tube assembly defines a cable opening which extends along the tube axis to receive a tip insert for performing the endoscopic surgery. The tube assembly defines a groove for receiving the lock to retain the tube assembly to the housing when the lock is disposed in the groove.

In a third aspect, the present invention provides a surgical instrument for performing endoscopic surgery. The surgical instrument includes a housing. A first handle is pivotally attached to the housing. A first ratchet member defines a rotational hole on a rotational axis with the first ratchet pivotally attached to the first handle for rotating about the rotational axis relative to the first handle. The first ratchet member includes at least one serrated tooth. A second handle is pivotally attached to the housing. A second ratchet member extends from the second handle. The second ratchet member includes a finger for engagement with the at least one serrated tooth of the first ratchet member to prevent movement of the first handle relative to the second handle. The first ratchet member is rotatable about the rotational axis to disengage the at least one serrated tooth from the finger to allow the first handle to move relative to the second handle.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a perspective view of a surgical instrument;

FIG. 2 is a exploded view of the surgical instrument of FIG. 1;

FIG. 3A is a cross-sectional side view of the surgical instrument of FIG. 1 showing a pair of handles in an opened position an a first ratchet member in a retracted “OFF” position;

FIG. 3B is a cross-sectional side view of the surgical instrument of FIG. 1 showing the pair of handles in a closed position with the first ratchet member in the retracted “OFF” position;

FIG. 3C is a cross-sectional side view of the surgical instrument of FIG. 1, taken along line 3C-3C, showing the pair of handles in the closed position with the first ratchet member in an active “ON” position with a finger of a second ratchet member engaging on of a plurality of serrated teeth of the first ratchet member;

FIG. 4 is a cross-sectional top view of the surgical instrument of FIG. 1, taken along line 4-4, showing a lock extending into a groove of a tube adaptor to retain a tube assembly in a body of the surgical instrument;

FIG. 5 is a cross-sectional view of the lock extending into the groove of the tube adaptor with a lever in a relaxed position;

FIG. 6 is a cross-sectional view of the lock disengaged from the groove of the tube adaptor with the lever in a depressed position;

FIG. 7 is a perspective view of the latch showing the tube adaptor, the lock, and the lever;

FIG. 8 is an exploded perspective view of the latch, the lock, the spring, and the lever;

FIG. 9 is a perspective view of an alternative embodiment of the tube adaptor, the lock, and the lever;

FIG. 10 is a cross-sectional end view of the first handle showing a release button in an “ON position” extending through the first handle and a release arm of the first ratchet member disposed against a ramp portion of the release button for rotating the first ratchet member;

FIG. 11 is a cross-sectional end view of the first handle showing the release button in an “OFF position” extending through the first handle with the release button rotated such that the release arm of the first ratchet member is not disposed against the ramp portion of the shaft;

FIG. 12 is a cross-sectional end view of the first handle showing the release button in the “ON position” extending through the first handle and a release arm of the first ratchet member disposed against a ramp portion of the release button with the release button depressed;

FIG. 13 is a partially exploded perspective view of the first handle, the release button, and a first ratchet member;

FIG. 14 is a cross-sectional top view of the surgical instrument of FIG. 1, taken along line 4-4, showing an alternative lock extending into a groove of a tube adaptor to retain a tube assembly in a body of the surgical instrument;

FIG. 15 is a cross-sectional view of the alternative lock of FIG. 14 extending into the groove of the tube adaptor with a lever in a relaxed position;

FIG. 16 is a cross-sectional view of the alternative lock of FIG. 14 disengaged from the groove of the tube adaptor with the lever in a depressed position;

FIG. 17 is a perspective view of the latch showing the tube adaptor, the lock, and the lever;

FIG. 18 is an exploded perspective view of the alternative latch of FIG. 14, the lock, and the lever; and

FIG. 19 is a perspective view of yet another embodiment of the tube adaptor, the lock, and the lever.

### DETAILED DESCRIPTION OF THE INVENTION

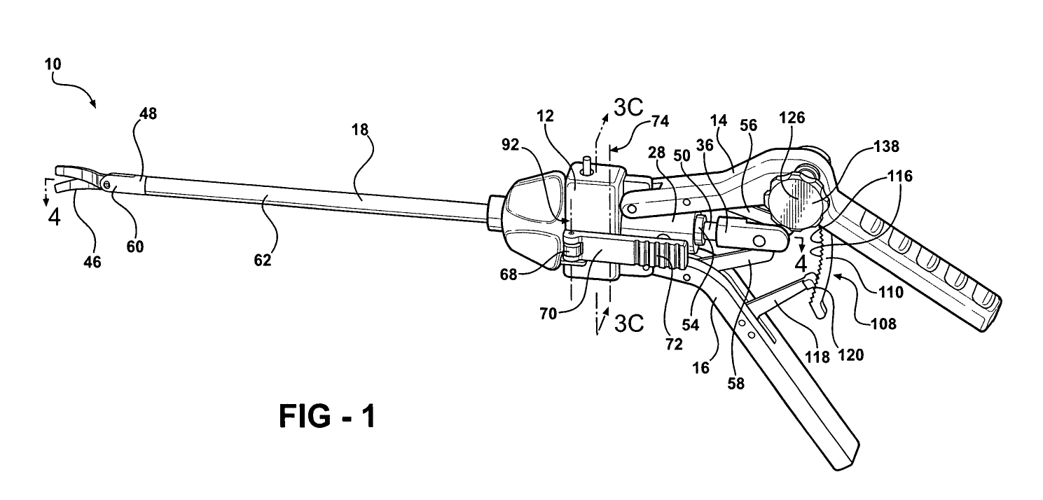

Referring to the Figures, wherein like numerals indicate corresponding parts throughout the several views, a surgical instrument 10 is generally shown at 10 in FIG. 1. The surgical instrument 10 is forceps or any other instrument for performing various procedures during endoscopic or laparoscopic types of surgery. A common type of procedure is cutting. However, the surgical instrument 10 can be used to perform other types of procedures such as grasping, manipulating, or ablating, for example. The surgical instrument 10 includes a housing 12, a pair of handles 14, 16, and a tube assembly 18. The handles 14, 16 are pivotally connected to the housing 12.

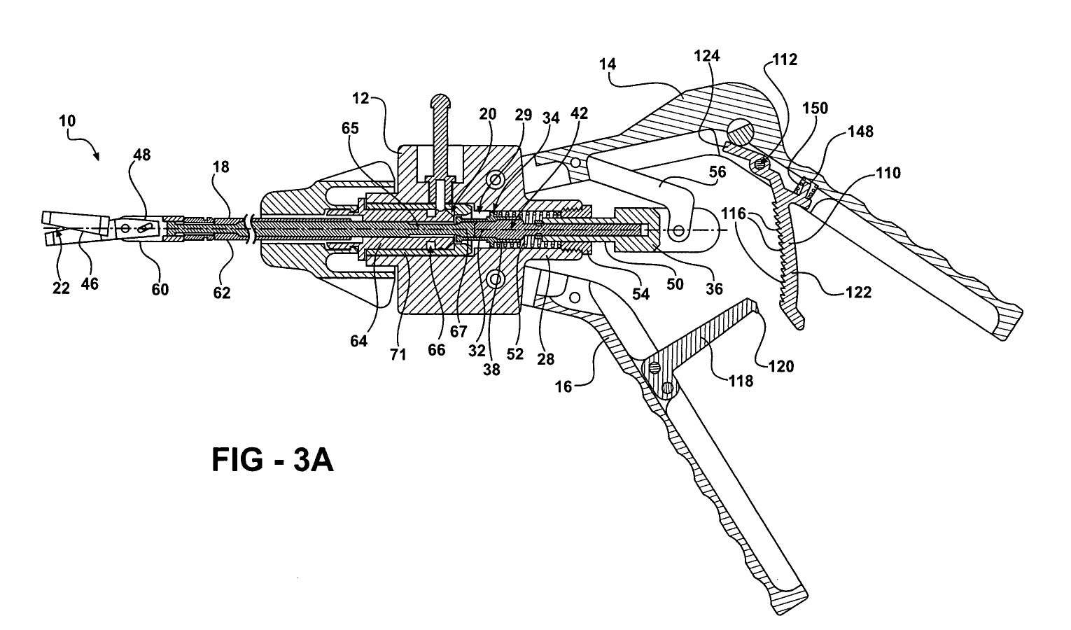

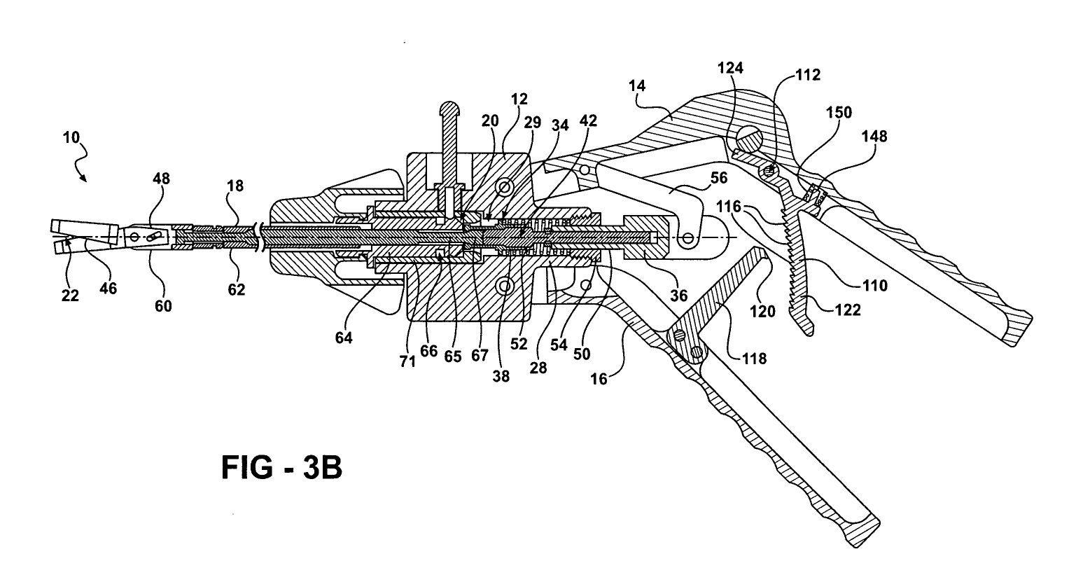

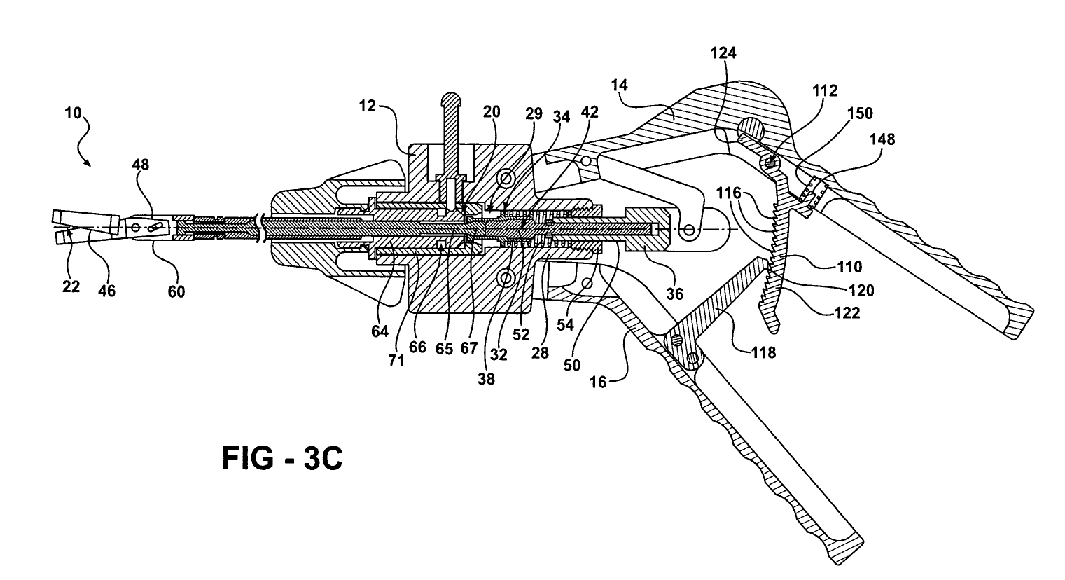

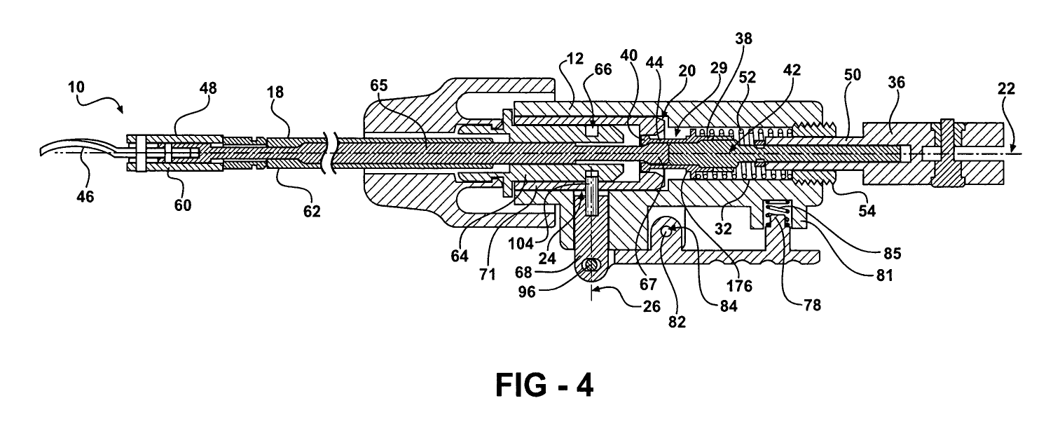

Referring to FIGS. 3A-3C and 4, the housing 12 defines an axial bore 20 extending along a tube axis 22. The housing 12 also defines a transverse bore 24 which extends along a transverse axis 26 and opens to the axial bore 20. Therefore, the transverse bore 24 is transverse to, and intersects, the axial bore 20. An hollow arm 28 extends from the housing 12. The hollow arm 28 defines a collet chamber 29 on the tube axis 22 with the collet chamber 29 opening to the axial bore 20.

A grabbing assembly 32 is disposed in the housing 12. The grabbing assembly 32 includes a collet 34 and a stem 36. The collet 34 includes a collet housing 38 and jaws 40 extending from the collet housing 38. The collet housing 38 is generally cylindrical and defines a stem opening 42 for receiving a portion of the stem 36. The stem opening 42 extends through the collet housing 38 and into the jaws 40. The jaws 40 include two or more fingers 44 for grabbing a cable end 67 of a cable 65. The fingers 44 are elastically connected to the collet housing 38. This means that when the fingers 44 are in a “relaxed” position, the fingers 44 may diverge from the collet housing 12, by pushing the cable end 67 into the collet housing 38. When the fingers 44 are disposed inside of the collet chamber 29, the fingers 44 are “restricted” by the collet chamber 29 such that they extend from the collet housing 38 in a generally parallel relationship for retaining the cable end 67 of the cable 65.

The stem 36 includes a stem shaft 50. When the grabbing assembly 32 is disposed in the collet chamber 29, a compression spring 52 surrounds the stem shaft 50, inside of the collet chamber 29. The compression spring 52 is a coil spring. However, it should be appreciated that the compression spring 52 is not limited to being a coil spring as any other spring known to those skilled in the art may be used. The compression spring 52 is positioned between the collet housing 38 and a threaded nut 54. Accordingly, as the grabbing assembly 32 is pulled rearward in the collet chamber 29, the compression spring 52 is compressed between the collect housing 38 and the threaded nut 54. When the grabbing assembly 32 is released, the compression spring 52 forces the grabbing assembly 32 to slide forward in the collet chamber 29 and bias the handles 14, 16 apart from one another. A top link 56 and a bottom link 58 are used to interconnect the stem 36 to the handles 14, 16 for moving the stem 36, along the tube axis 22, in response to moving the handles 14, 16.

Loading and unloading the tube assembly 18 from the housing 12 is facilitated by spreading the upper and lower handles 14, 16 far apart, i.e., pulled apart from one another, such that the links 56, 58 cause the stem 36 to push the fingers 44 and and cable end 67 all the way into the axial bore 20. When the fingers 44 of the collet 38and the cable end 67 of the collet 34 enter the axial bore 20, the fingers 44 and cable end 67 are no longer restricted by the collet chamber 29 and no longer secure or engage the cable end 67, if a tube assembly 18 is already loaded into the housing 12.

As the upper and the lower handles 14, 16 are closed, i.e., pulled toward one another, the links 56, 58 cause the stem 36 to pull the fingers 44 and the cable end 67 into the collet chamber 29. As the cable end 67 is grabbed, it pulls, and retains, the cable end 67 of the cable 65 of a tip assembly 48 if the tip assembly 48 is inserted into the housing 12.

Similarly, movement of the handles 14, 16 relative to one another moves a blade or jaws 46 relative to a tip 60 in the tip assembly 48. However, the handles 14, 16 are not spread as far apart as when loading and unloading the tube assembly 18 from the housing 12. Therefore, when the handles 14, 16 are moved apart, the links 56, 58 cause the stem 36 to push the fingers 44 forward in the collet chamber 29 which, in turn, pushes the cable 70 forward through the tube 62. However, the blade or jaws 46 pivot within the tip assembly 48 in response to movement of the cable 65 along the tube axis 22. The surgical instrument 10 is not limited to using the tip assembly 48 as shown in the Figures. It should be appreciated that the present invention allows for interchangeability between any desired number of tip assemblies 48 for any desired number of surgical applications.

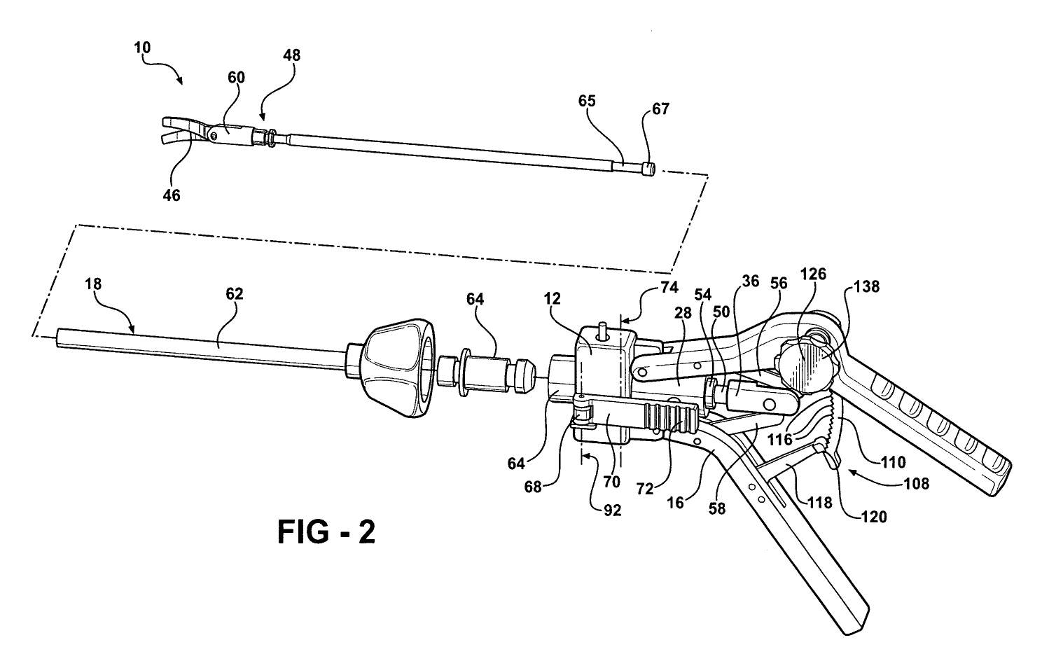

Referring generally to FIG. 2, the tube assembly 18 is removably disposed in the axial bore 20 of the housing 12. The tube assembly 18 includes a hollow tube 62 extending from a tube adaptor 64. The tip assembly 48 is inserted into the hollow tube 62 and the tube adaptor 64. Any desired tip assembly 48 can be inserted into the cable opening 66, such as for cutting, ablating, or for any other desired function. The tube assembly 18 includes the tube adaptor 64. In one embodiment, the tube adaptor 64 defines a groove 66 extending circumferentially about the tube adaptor 64 for receiving a lock 68 to retain the tube assembly 18 to the housing 12 when the lock 68, as shown in FIG. 4, is disposed in the groove 66 within the axial bore 20. Additionally, a wear cylinder 71 is disposed in axial bore 20 of the housing 12 between the adaptor 64 and the housing 12. The wear cylinder 71 is formed of metal, or any other material for preventing or inhibiting frictional wear.

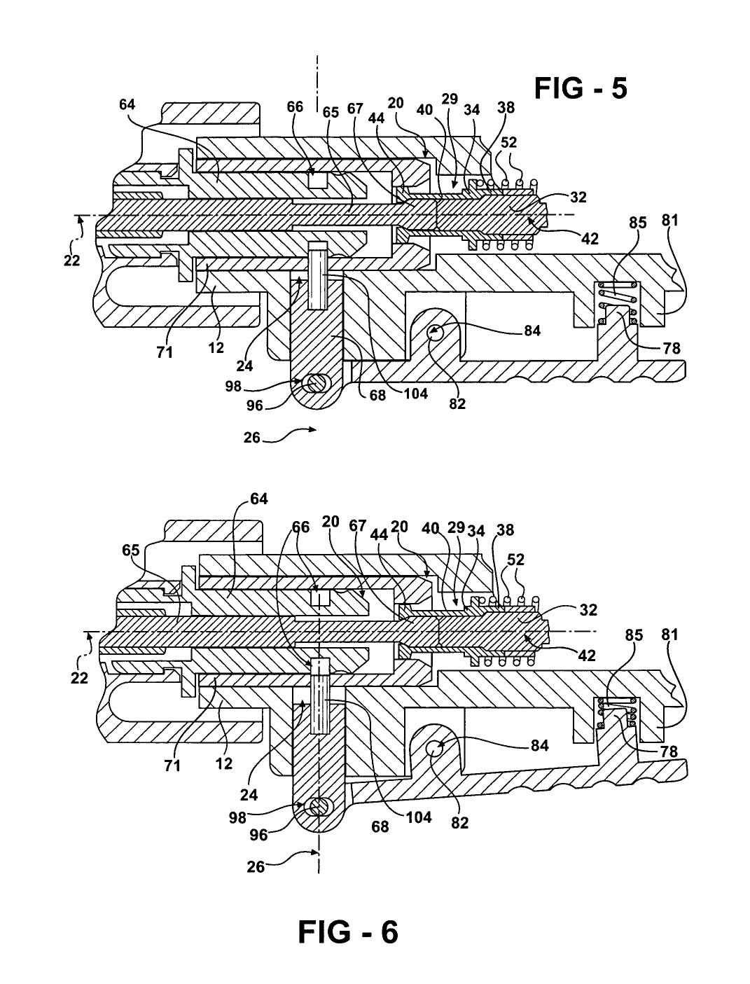

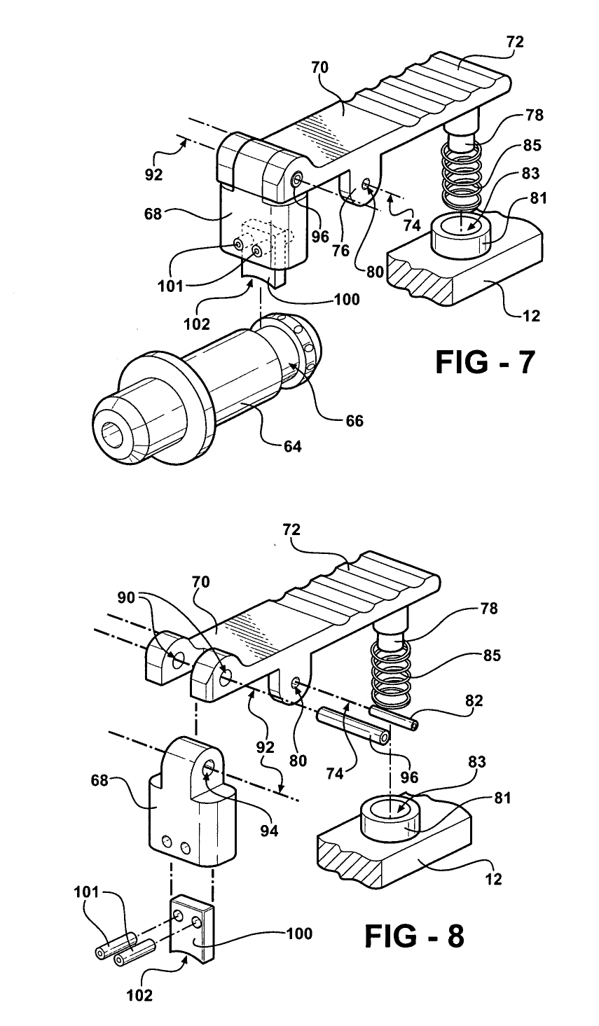

Referring to FIGS. 4-6, the lock 68 is slidably disposed in the transverse bore 24 of the housing 12, along the transverse axis 26. This means that the lock 68 slides inward and outward along the transverse bore 24 with respect to the housing 16. The lock 68 moves into the axial bore 20 to engage the groove 66 of the tube adaptor 64. Alternatively, the lock 68 moves out of the axial bore 20 and into the transverse bore 24 to disengage the groove 66 of the tube adaptor 64. The lock 68 includes a locking piece 100 with the locking piece 100 disposed in the axial bore 20 for engaging the tube adaptor 64. The locking piece 100 is formed as a piece which is inserted into the lock 68 and attached with fasteners 101, as shown in FIGS. 7-9. However, the locking piece can be attached to the lock 68 in any desired fashion, such as by adhesive, welding, etc. Alternatively, the locking piece 100 and the lock 68 are formed as a single component. The locking piece 100 defines an arced groove 102 having a concave and arced shape. The arced groove 102 is conformed to ride in the groove 66 of the tube adaptor 64 as the tube adaptor 64 rotates within the axial bore 20 about the tube axis 22 while engaging and retaining the tube adaptor 64 within the axial bore 20 of the housing 12. Referring to the embodiments shown in FIGS. 9 and 19, the lock 68 includes a locking pin 104 for engaging a locking hole 106 defined in the groove of the tube adaptor 64. The locking pin 104 is for locking the tube adaptor 64 to prevent rotation of the tube adaptor 64 within the axial bore 20. This allows the surgeon to set and lock 68 the instrument into a desired radial position.

A latch 70 extends from the lock 68 to an end portion 72, external to the transverse bore 24. The latch 70 defines a pivot axis 74 for pivoting the latch 70 about the pivot axis 74 as the lock 68 slides into or out of the axial bore 20. Referring again to FIG. 1, an underside of the latch 70 rests on the housing 12. As the end portion 72 of the latch 72 is depressed, i.e., moved toward the hollow arm 28, the latch 72 pivots about the pivot axis 74 on an edge of the housing 12.

Referring to FIGS. 4-9, the latch 70 includes a fulcrum 76. The fulcrum 76 defines the pivot axis 74 and a pivot hole 80 on the pivot axis 74. The housing 12 defines a second pivot hole 84 on the pivot axis 74. A first pin 82 extends through the first and second pivot holes 80, 84, along the pivot axis 74, for rotatably supporting the latch 70 as the latch 70 pivots about the pivot axis 74 relative to the housing 12 and to retain the latch 70 to the housing 12. A latch projection 78 extends from the latch 70, toward the housing 12. The housing 12 includes a protrusion 81 in alignment with the latch projection 78. The protrusion 81 defines a spring hole 83. A latch spring 85 is disposed about the latch projection 78 and inside of the spring hole 83 of the protrusion 81. Therefore, the latch spring 85 is disposed between the latch 70 and the housing 12. The protrusion 81 and the spring hole 83 retain the latch spring 85 between the latch 70 and the housing 12. As the latch 70 is pressed toward the housing 12, the latch spring 85 is compressed to move the locking piece 100 of the lock 68 out of the groove 66 of the tube adaptor 64. When the latch 70 is release, the latch spring 85 biases the latch 70 away from the housing 12, moving the locking piece 100 into the groove 66 of the tube adaptor 64. The latch spring 85 is not limited to being a coil spring, but may be any type of compressible spring the compresses under pressure to bias the latch 70 away from the housing 12 when pressure is released from the latch 70.

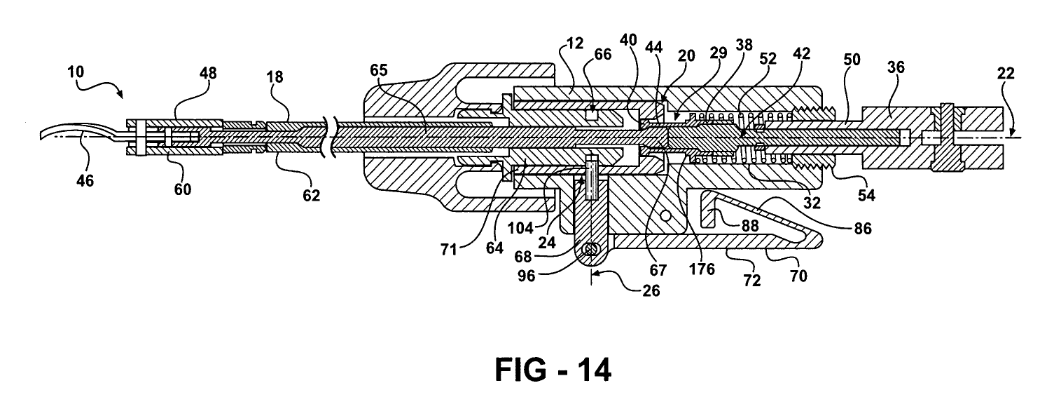

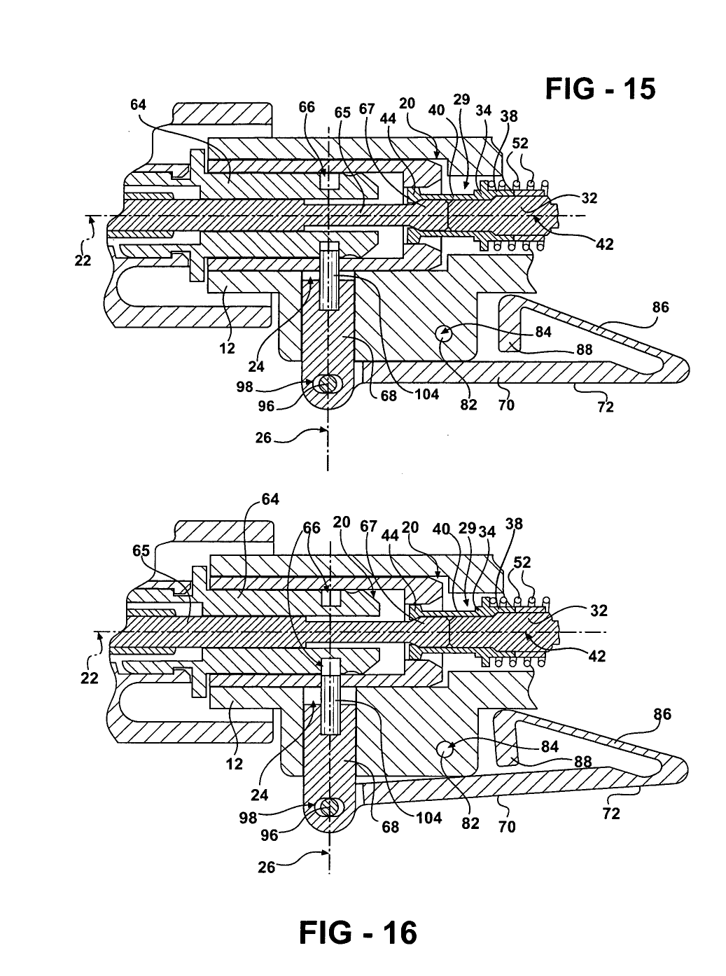

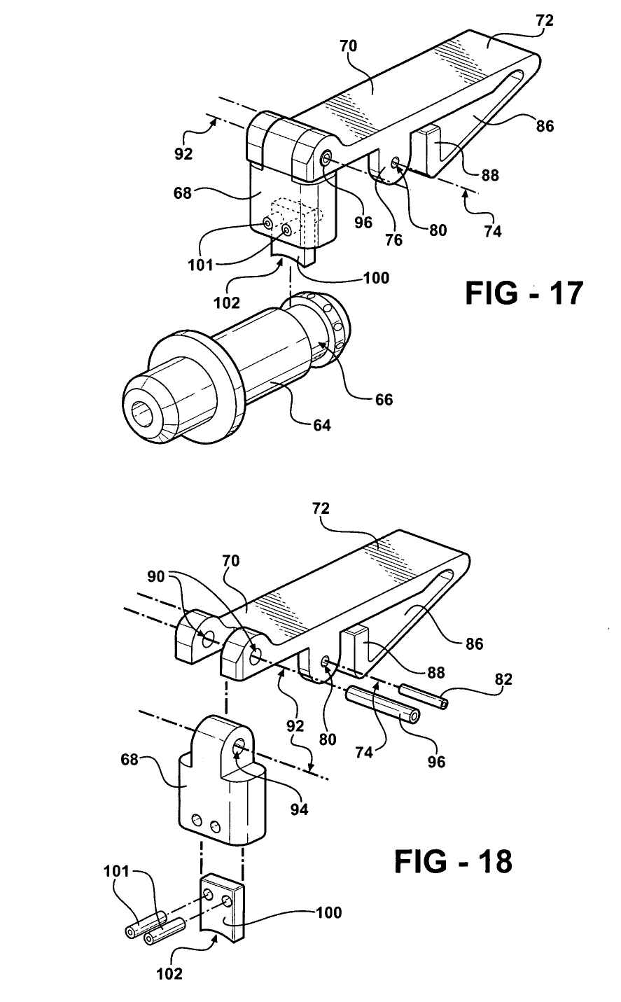

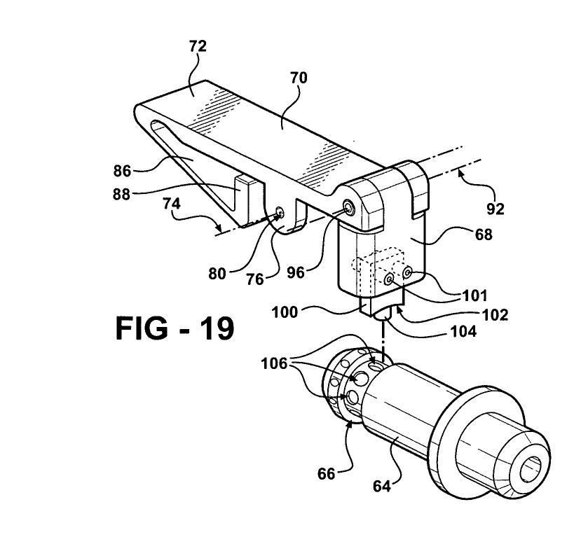

Referring to FIGS. 14-19, an alternative embodiment of the latch 70 is shown. In the alternative embodiment, a spring portion 86 extends from the end portion 72 of the latch 70. The spring portion 86 is disposed against the housing 12 and reacts between the latch 70 and the housing 12 to bias the lock 68 into the axial bore 20 of the housing 12 to engage the groove 66 of the tube adaptor 64. The spring portion 86 resiliently extends from the end portion 72. The spring portion 86 extends from the latch 70 in a V-shape. The spring portion 86 is disposed between the latch 70 and the housing 12. As pressure is applied to the end portion 72 of the latch, the spring portion 86 compresses and moves toward the latch 70 to collapse the V-shape. This compression moves the end portion 72 of the latch 70 toward the housing 12 and also moves the lock 68 out of the axial bore 20 to disengage the groove 66 of the tube adaptor 64. Conversely, when pressure is released from the end portion 72 of the latch 70, the spring portion 86 automatically moves away from the latch 70. As a result, the end portion 72 of the latch 70 moves away from the housing 12 and the lock 68 moves back into the axial bore 20, engaging the groove 66 of the tube adaptor 64. A spring stop 88 extends from the spring portion 86. The spring stop 88 is disposed between the spring portion 86 and the latch 70 for limiting movement of the spring portion 86 toward the latch 70 as the spring portion 86 is compressed. Therefore, the spring stop 88 limits the amount the V-shape is collapsed.

The latch 70 movably extends from the lock 68, as a hinged connection. The hinged connection allows the lock 68 to move along the transverse axis 26 as the latch 70 rotates about the pivot axis 74. The latch 70 defines a pair of first holes 90 on a hinge axis 92, spaced from the pivot axis 74. The lock 68 also defines a pair of second holes 94 also on the hinge axis 92. It should be appreciated, however, that more or less of the first and second holes 90, 94 may be used as desired. A second pin 96 extends through the first and second holes 90, 94 on the hinge axis 92. As the end portion 72 of the latch is moved toward the housing 16 and/or the hollow arm 28, pivoting the latch about the pivot axis 74, the lock 68 pivots about the hinge axis 92 relative to the latch 70. Additionally, as the end portion 72 of the latch is moved toward the housing 16 and/or the hollow arm 28, pivoting the latch about the pivot axis 74, the hinge axis 92 moves outward and away from the housing 16, moving the lock 68 outward along the transverse bore 24. Additionally, the first hole 90 is a slot 98 and the second pin 96 moves or slides along the slot 98 as the lock 68 pivots relative to the latch 70 about the hinge axis 92. However, the slot-and-hole arrangement may be reversed where the first hole 90 is the slot 98.

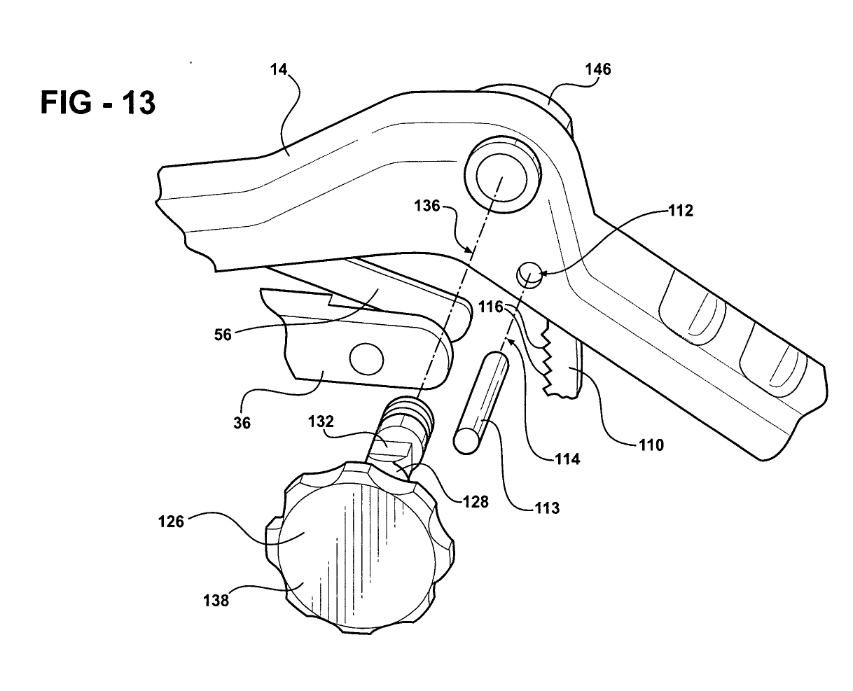

Referring to FIGS. 1, 2, 3A-3C, and 13, the surgical instrument 10 also includes a ratcheting feature 108 for limiting movement of the first handle 14 relative to the second handle 16 when the surgeon is performing certain procedures. For example, if the surgeon is clamping, the surgeon draws the handles together and handles are prevented from automatically moving away from one another. The ratcheting feature 108 includes a first ratchet member 110 defining a rotational hole 112 on a rotational axis 114, as shown in FIG. 13, with the first ratchet member 110 pivotally attached to the first handle 14 for rotating about the rotational axis 114 relative to the first handle 14 and a second ratchet member 118 extending from the second handle 16. A rotational pin 113 is inserted through the rotational hole 112, on the rotational axis 114, to retain the first ratchet member 110 to the first handle 14 while allowing the first ratchet member 110 to rotate about the rotational axis 114. The first ratchet member 110 includes at least one serrated tooth 116. However, any desired number of serrated teeth 116 may be used such that the finger 120 moves, or ratchets, along and individually engages the serrated teeth 116 as the first handle 14 moves toward the second handle 16 to lock the first handle 14 to relative to the second handle 16. The second ratchet member 118 is in fixed relationship to the second handle 16. The ratchet feature also includes a second ratchet member 118. The second ratchet member 118 includes a finger 120 for engagement with one of the serrated teeth 116 of the first ratchet member 110. This engagement temporarily prevents movement of the first handle 14 relative to the second handle 16. The first ratchet member 110 is rotatable about the rotational axis 114 to selectively disengage the finger 120 from the serrated teeth 116 to restore movement of the first handle 14 relative to the second handle 16.

The first ratchet member 110 is curved and includes a ratchet portion 122 with the serrated teeth 116 formed on the ratchet potion. The first ratchet member 110 also includes a release arm 124. The rotational hole 112 is defined between the release arm 124 and the ratchet portion 122 for applying a rotational force to the release arm 124 to rotate the first ratchet member 110 about the rotational axis 114 to disengage the finger 120 from the serrated tooth 116.

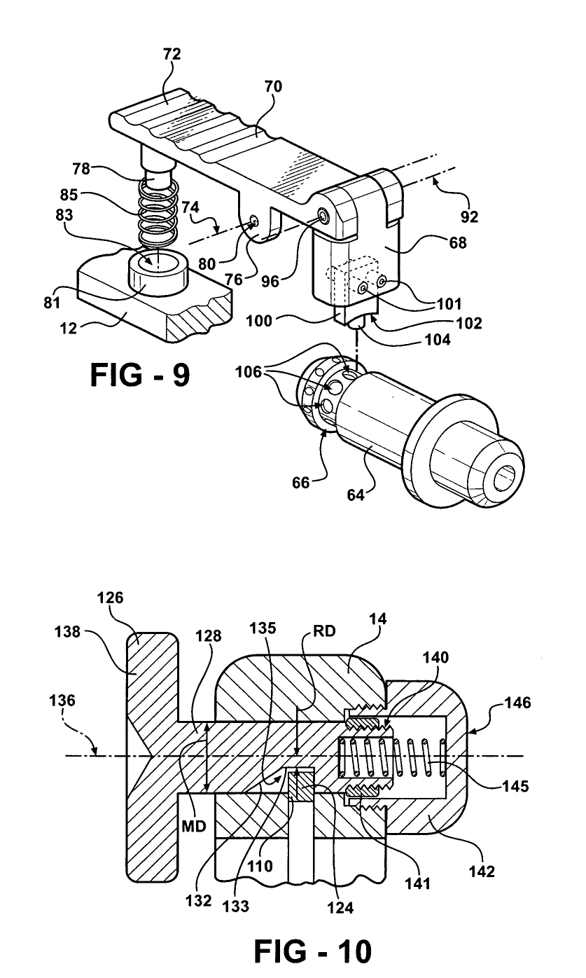

A release button 126 is disposed on the first handle 14 adjacent to the release arm 124 of the first ratchet member 110 for applying the rotational force to the release arm 124 to rotate the first ratchet member 110 about the rotational axis 114 to disengage the finger 120 from the serrated tooth 116. Referring to FIGS. 10-12, the release button 126 includes a shaft 128 which has a major thickness MD. The shaft 128 defines a channel 135. A ramp portion 132 extends into the channel 135 which decreases the width from the major thickness MD to a reduced thickness RD which is less than the major thickness MD. The ramp portion 132 decreases along the ramp portion 132 to the reduced thickness RD at a flat portion 133, extending along the shaft 128 at a generally constant thickness. Typically, the shaft 128 of the release button 126 extends through the first handle 14. The shaft 128 extends along a release axis 136. The release button 126 is movable along the release axis 136. The release button 126 applies the required rotational force to the release arm 124 to move the serrated tooth 116 or teeth 116 away from the finger 120. This happens when the release arm 124 is moved along the ramp portion 132 from the reduced thickness RD to approximately the major thickness MD. Conversely, as the rotational force is removed from the release arm 124, the release arm 124 is moved along the ramp portion 132 from the major thickness MD to the reduced thickness RD to move the serrated tooth 116 or teeth 116 toward and into engagement with the finger 120.

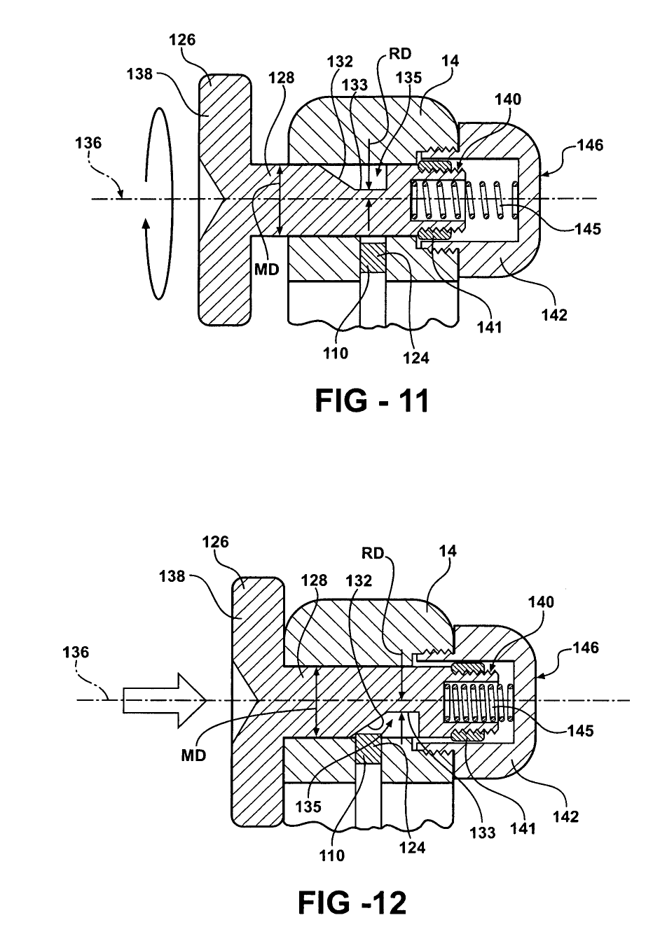

The release button 126 is also rotatably disposed on the first handle 14 about the release axis 136 for rotating the release button 126 about the release axis 136 to a ratcheting position, or “ON position”, as shown in FIG. 10. When in the “ON position,” the release arm 124 is disposed against the ramp portion 132. When the release button 126 is rotated about the release axis 136 to a non-ratcheting position, or “OFF position”, as shown in FIG. 11, the release arm 124 is disposed against only the major thickness MD or some other diameter which is adequate to move the first ratchet member 110 out of engagement with the second ratchet member 118. As the release arm 124 moves along the ramp portion 132 between the reduced thickness RD and the major thickness MD or rests completely on the shaft 128, the first ratchet member 110 rotates about the rotational axis 114. As the release button 126 is turned about the release axis 136, the release button 126 is moved into one of two possible positions.

The release button 126 also includes a head 138 for moving the release button 126 either along the release axis 136, as shown in FIG. 12, or about the release axis 136, as shown in FIGS. 10 and 11. The release button 126 defines external threads 140. The shaft 128 of the release button 126 is inserted through the first handle 14. The external threads 140 are threaded into a locking nut 141 to retain the release button 126 to the first handle 14. An external nut 142 is threaded into the first handle 14, opposite the head 138 and a release spring 145 is disposed between the external nut 142 and the shaft 128. The release spring 145 biases the shaft of the release button 126 along the release axis 136 and away from the external nut 142. The external nut 142 encases the locking nut 141 and the external threads 140. The external nut 142 provides a surface 146 which opposes the head 138 for allowing the head 138 to be depressed with the surgeon's finger, such as a thumb, along the release axis 136 for one-handed use. When the release button 126 is in the “ON position” as the release button 126 moves along the release axis 136, toward the external nut 142, the locking nut 141 and a portion of the shaft 128 enter into the external nut 142 and the ramp portion moves the first ratchet member 110 away from the finger 120 of the second ratchet member 118. Conversely, as pressure from the surgeon is reduced on the head 138, the release button 126 is biased by the release spring 145 and moves along the release axis 136, away from the external nut 142 and the release arm 124 of the first ratchet member 110 moves down the ramp portion 132 to bring the first ratchet member into engagement with the finger 120 of the second ratchet member 118.

The first ratchet member 110 also includes a spring member 148 for biasing the first ratchet member 110 about the rotational axis 114 and away from the first handle 14 and into engagement with the finger 120 of the second ratchet member 118. A reaction spring 150 is disposed on the first handle 14. The spring member 148 reacts against the reaction spring 150 for biasing the first ratchet member 110 about the rotational axis 114 and away from the first handle 14 and into engagement with the finger 120 of the second ratchet member 118. The ratchet spring 150 is a coil spring. It should be appreciated, however, that the ratchet spring 150 is not limited to being a coil spring, but may be any type of spring known to those skilled in the art which is suitable for reacting between the spring member 148 and the first handle 14.

Many modifications and variations of the present invention are possible in light of the above teachings. In addition, the reference numerals in the claims are merely for convenience and are not to be read in any way as limiting.

What is claimed is:

1. A body assembly of a surgical instrument for performing endoscopic surgery, said handle assembly comprising:
a housing defining an axial bore;
said housing defining a transverse bore extending along a transverse axis and opening to said axial bore;
a lock slidably disposed in said transverse bore along said transverse axis for sliding into said axial bore to engage a tube adaptor and for sliding out of said axial bore to disengage the tube adaptor;
a latch extending from said lock to an end portion; and
said latch defining a pivot axis for pivoting said latch about said pivot axis as said lock slides into or out of said axial bore.

2. A body assembly as set forth in claim 1 further comprising a spring portion disposed between said latch and said housing for reacting between said latch and said housing to bias said lock into said axial bore.

3. A body assembly as set forth in claim 2 wherein said spring portion is defined as a coil spring.

4. A body assembly as set forth in claim 2 wherein said spring portion is further defined as extending from said end portion with said spring portion being disposed against said housing for reacting between said latch and said housing to bias said lock into said axial bore.

5. A body assembly as set forth in claim 4 wherein said spring portion is further defined as resiliently extending from said end portion.

6. A body assembly as set forth in claim 5 wherein said spring portion is further defined as extending from said latch in a V-shape with said spring portion disposed between said latch and said housing for compressing said spring portion to move said spring portion toward said latch as pressure is applied to said end portion of said latch to move said end portion of said latch toward said housing and said lock out of said axial bore and to automatically move said spring portion away from said latch a pressure is released from said end portion of said latch to move said end portion of said latch away from said housing and said lock into said axial bore.

7. A body assembly as set forth in claim 6 further comprising a spring stop extending from said spring portion with said spring stop disposed between said spring portion and said latch for limiting movement of said spring portion toward said latch as said spring portion is compressed.

8. A body assembly as set forth in claim 1 wherein said latch is further defined as movably extending from said lock for allowing said lock to move along said transverse axis as said latch rotates about said pivot axis.

9. A body assembly as set forth in claim 8 wherein said latch defines at least one first hole on a hinge axis and said lock defines at least one second hole on said hinge axis and further comprising a second pin extending through said first and second holes on said hinge axis such that said latch pivots about said pivot axis as said end portion is moved toward said housing and said lock pivots relative to said latch about said hinge axis.

10. A body assembly as set forth in claim 9 wherein said at least one first hole is further defined as a slot and said pin moves along said slot as said lock pivots relative to said latch about said hinge axis.

11. A body assembly as set forth in claim 1 wherein said lock includes a locking piece with said locking piece disposed in said axial bore for engaging the tube adaptor.

12. A body assembly as set forth in claim 11 wherein said locking piece defines an arced groove having a concave and arced shape for allowing the tube adaptor to rotate within the axial bore of the housing while engaging the tube adaptor.

13. A body assembly as set forth in claim 1 wherein said lock includes a locking pin for engaging a locking hole defined in the tube adaptor for locking the tube adaptor to prevent rotation of the tube adaptor within said axial bore.

14. A body assembly as set forth in claim 1 wherein said latch includes a fulcrum with said fulcrum defining said pivot axis.

15. A body assembly as set forth in claim 14 wherein said fulcrum defines a pivot hole on said pivot axis and further comprising a pin extending along said pivot axis for rotatably supporting said latch as said latch pivots about said pivot axis relative to said housing.

16. A body assembly as set forth in claim 15 wherein said housing further defines a second pivot hole on said pivot axis.

17. A surgical instrument for performing endoscopic surgery, said surgical instrument comprising:
a housing defining an axial bore extending along a tube axis,
said housing defining a transverse bore extending along a transverse axis and opening to said axial bore,
a lock slidably disposed in said transverse bore along said transverse axis for sliding into said axial bore to engage a tube adaptor and for sliding out of said axial bore to disengage the tube adaptor,
a latch extending from said lock to an end portion with said latch defining a pivot axis for pivoting said latch about said pivot axis as said lock slides into or out of said axial bore;
a tube assembly defining a cable opening extending along said tube axis to receive a tip insert for performing the endoscopic surgery; and
said tube assembly defining a groove for receiving said lock to retain said tube assembly to said housing when said lock is disposed in said groove.

18. A surgical instrument for performing endoscopic surgery, said surgical instrument comprising:
a housing;
a first handle pivotally attached to said housing;
a first ratchet member defining a rotational hole on a rotational axis with said first ratchet pivotally attached to said first handle for rotating about said rotational axis relative to said first handle;
said first ratchet member including at least one serrated tooth;
a second handle pivotally attached to said housing;
a second ratchet member extending from said second handle;
said second ratchet member including a finger for engagement with said at least one serrated tooth of said first ratchet member to prevent movement of said first handle relative to said second handle; and
said first ratchet member rotatable about said rotational axis to disengage said at least one serrated tooth from said finger to allow said first handle to move relative to said second handle.

19. A surgical instrument as set forth in claim 18 wherein said first ratchet member is curved.

20. A surgical instrument as set forth in claim 18 wherein said first ratchet member includes a ratchet portion with said at least one serrated tooth formed on said ratchet potion.

21. A surgical instrument as set forth in claim 20 wherein said at least one serrated tooth is further defined as a plurality of serrated teeth disposed along said ratchet portion such that said finger moves along and individually engages said plurality of serrated teeth as said first handle moves toward said second handle to lock said first handle to relative to said second handle.

22. A surgical instrument as set forth in claim 19 wherein said first ratchet member further includes a release arm with said rotational hole defined between said release arm and said ratchet portion for applying a rotational force to said release arm to rotate said first ratchet member about said rotational axis to disengage said finger from said serrated tooth.

23. A surgical instrument as set forth in claim 22 further comprising a release button disposed on said first handle adjacent said release arm of said first ratchet member for applying said rotational force to said release arm to rotate said first ratchet member about said rotational axis to disengage said finger from said serrated tooth.

24. A surgical instrument as set forth in claim 23 wherein said release button includes a shaft defining a channel with a ramp portion extending along the channel from a major thickness and decreasing to a reduced thickness with said shaft extending along said release axis to apply said rotational force to release arm and move said serrated tooth away from said finger when said release arm is moved along said ramp portion from said reduced thickness to said major thickness and to remove said rotational force from said release arm and move said serrated tooth toward and into engagement with said finger when said release arm is moved along said ramp portion from said major thickness to said reduced thickness.

25. A surgical instrument as set forth in claim 24 wherein said release button further includes a flat portion extending from said ramp portion along said shaft at said reduced thickness.

26. A surgical instrument as set forth in claim 24 wherein said release button is further defined as being rotatably disposed on said first handle about said release axis for rotating said release button about said release axis to a ratcheting position such that said release arm is disposed against said ramp portion and for rotating said release button about said release axis to a non-ratcheting position such that said release arm is disposed against only said major thickness.

27. A surgical instrument as set forth in claim 24 wherein said release button further includes a head for applying a force to said release button.

28. A surgical instrument as set forth in claim 20 wherein said first ratchet member further includes a spring member for biasing said first ratchet member about said rotational axis and away from said first handle and into engagement with said finger of said second ratchet member.

29. A surgical instrument as set forth in claim 28 wherein said spring member extends from said ratchet portion.

30. A surgical instrument as set forth in claim 29 further comprising a reaction spring disposed on said first handle with said spring member reacting against said reaction spring for biasing said first ratchet member about said rotational axis and away from said first handle and into engagement with said finger of said second ratchet member.
